(19) United States
(12) Patent Application Publication
(10) Pub. No.: US 2026/0208841 A1
(43) Pub. Date: Jul. 23, 2026

(54) RESONATING SYSTEM - THE LIMP

(71) Applicant: ROBEAUTÉ, Paris (FR)

(72) Inventors: Bertrand DUPLAT, Paris (FR); Ali OULMAS, Paris (FR); Quentin FRANCOIS, Paris (FR)

(73) Assignee: ROBEAUTÉ, Paris (FR)

(21) Appl. No.: 19/136,955

(22) PCT Filed: Dec. 6, 2023
(86) PCT No.: PCT/EP2023/084614

§ 371 Date: Jun. 9, 2025

(30) Foreign Application Priority Data
Dec. 9, 2022 (EP) 22306833.9

(51) Int. Cl. B63H 21/00; A61B 34/30; G05D 1/467; G05D 107/50

(52) U.S. Cl. CPC B63H 21/00; A61B 34/30; G05D 1/467; G05D 2107/50

(57) ABSTRACT

Micro-robot including a body configured to vibrate, and an actuator configured to generate vibrations causing the micro-robot to move. It includes a steering system which includes a resonating structure configured to be secured to the micro-robot and including a steering structure to control the propulsion direction, weight-resonators being configured to be activated by a proper activation resonance frequency. The actuator is configured to generate vibrations in a range of frequencies including the proper activation resonance frequency of each weight-resonator. The resonating structure displays at least two states: one activated steering state, in which at least one of the weight-resonators is activated at the proper activation resonance frequency to change the propulsion direction, and a non-activated steering state, in which none of the weight-resonators is activated at its proper activation resonance frequency so as to maintain the propulsion direction.

### FIELD

The present invention relates to a steering system of a micro-robot aimed at circulating through a viscous environment, in particular inside a human body.

### BACKGROUND

The ability to reach deep and functional structures without damage is a major challenge in mini-invasive surgery, especially in neurosurgery. Thanks to microtechnologies, it becomes possible to send a fully autonomous microrobot inside an organ of a subject, such as a brain. However, the propulsion of a microrobot in an environment at low Reynolds number, as in the brain, is a challenge because of absence of inertia and presence of relatively high drag forces due to the small size of the microrobot. Another important requirement is that the microrobot should be capable of moving in an organ while limiting as much as possible the physiological damage that its passage causes to the organ.

In this context, the invention is intended to propose a microrobot having a highly efficient propulsion mechanism in a viscous environment at low Reynolds number, while preserving as much as possible the integrity of the environment in which it is displaced.

### SUMMARY

This invention thus relates to a micro-robot configured to move along a propulsion direction by vibrations, said micro-robot comprising a body configured to vibrate, and an actuator configured to generate vibrations causing the micro-robot to move, said micro-robot further comprising a steering system which comprises a resonating structure configured to be secured to the micro-robot, the resonating structure comprising:
a steering structure aimed at controlling the propulsion direction,
a distribution of weight-resonators, each weight-resonator being configured to be activated by a proper activation resonance frequency, the respective proper activation resonance frequencies of the weight-resonators being different from each other,
wherein the actuator is configured to generate vibrations in a range of frequencies including the proper activation resonance frequency of each weight-resonator,
wherein the resonating structure displays at least two states:
   at least one activated steering state, in which at least one of the weight-resonators is activated at the proper activation resonance frequency to change the propulsion direction of the micro-robot,
   a non-activated steering state, in which none of the weight-resonators is activated at its proper activation resonance frequency so as to maintain the propulsion direction of the micro-robot.

Thus, this solution achieves the above objective. In particular, it allows the obtaining of a rotation of the micro-robot solely based on the movements (energy) generated by the micro-robot itself (more precisely by the micro-motor of the micro-robot), thus avoiding the addition of extra-energy which might lead to further elements or devices to be added inside the patient or additional energy to be conveyed to the micro-robot, all which might lead to increase risks of damaging the environment in which the micro-robot moves.

The device according to the invention may include one or more of the following characteristics, taken in isolation from one another or in combination with one another:
the resonating structure may display several activated steering states, each activated steering state being associated to a different proper activation resonance frequency,
   the at least one activated steering state, is a state in which the configuration and movement of the resonating structure aim at changing the propulsion direction of the micro-robot,
   the non-activated steering state, is a state in which the configuration and movement of the resonating structure aim at maintaining the propulsion direction of the micro-robot,
the resonating structure may further comprise a distribution of multi-stable elements, each multi-stable element being deformable between at least two stable configurations:
   at least a first stable configuration when the resonating structure is in its activated steering state,
   at least a second stable configuration when the resonating structure is in its non-activated steering state,
the multi-stable element may be a bi-stable pre-compressed beam displaying the first and the second stable configurations, the pre-compressed beam being bent in a first direction towards the body of the micro-robot in the first stable configuration and being bent in a second direction away from the body of the micro-robot in the second stable configuration,
the steering structure may be a retractable steering foil displaying an open and a retracted configuration with regards to the body of the micro-robot, the steering foil configuration being determined by the steering state of the resonating structure:
   the non-activated steering state of the resonating structure induces the retracted configuration of the retractable steering foil, and
   the activated steering state of the resonating structure induces the open configuration of the retractable steering foil,
each multi-stable element may be a multi-stable shell comprising a stack of several sheets,
the steering system may further comprise at least one mobile cilium, the at least one cilium being configured to be put in motion by the vibration of the body of the micro-robot,
the resonating structure may comprise at least one strand of a propulsion spring comprised within the body,
the activation of the resonating structure in its at least one activated steering state may induce the retractation of the at least one strand of the propulsion spring,
the body of the micro-robot may present a global circular symmetry along a propulsion axis parallel to the propulsion direction, wherein the resonating structure is further part of said body, and wherein the activation of the resonating structure into one of its activated steering states induces a break in the global circular symmetry of the body,
the at least one cilium may be part of the resonating structure, the at least one cilium presenting a first movement intensity in the non-activated steering state and a second movement intensity in the activated steering state, the second movement intensity being different than the first movement intensity,
each cilium may comprise a weight-resonator.

### BRIEF DESCRIPTION OF THE DRAWINGS

The invention will be better understood, and other aims, details, characteristics and advantages thereof will emerge more clearly on reading the detailed explanatory description which follows, of embodiments of the invention given by way of illustration. purely illustrative and non-limiting examples, with reference to the accompanying drawings.

FIG. 1a is a general schematic side view of the micro robot comprising a steering system according to a first embodiment of the present invention,

FIG. 1b is a general schematic side view of the micro robot comprising a steering system according to a second embodiment of the present invention,

FIG. 2a is a detailed schematic side view of the steering system according to the first embodiment of the present invention,

FIG. 2b is a detailed schematic side view of the steering system according to the second embodiment of the present invention,

FIG. 3a is a detailed schematic view from above of the steering system according to the first embodiment of the present invention,

FIG. 3b is a detailed schematic view from above of the steering system according to the second embodiment of the present invention,

FIG. 4 is a step-by-step schematic view of the activation of the steering system according to the first embodiment,

FIG. 5a is a general schematic side view of the micro robot comprising a steering system according to a third embodiment of the present invention,

FIG. 5b is a detailed schematic side view of the steering system according to the third embodiment of the present invention,

FIG. 5c is a detailed schematic view from above of the steering system according to the third embodiment of the present invention,

FIG. 6 is a step-by-step schematic view of the activation of the steering system according to the third embodiment of the present invention,

FIG. 7a is a general schematic side view of the micro robot comprising a steering system according to a fourth embodiment of the present invention,

FIG. 7b is a detailed schematic side view of the steering system according to the fourth embodiment of the present invention,

FIG. 7c is a detailed schematic view from above of the steering system according to the fourth embodiment of the present invention,

FIG. 8 is a step-by-step schematic view of the activation of the steering system according to the fourth embodiment of the present invention.

### DETAILED DESCRIPTION

Please note that in the present application, the term “weight-resonator” is given a wide definition: It is not necessarily just resonators based on their weight. They might also include foils and bi-stable or multi-stable elements which enable to play with the shape of the part and the internal tensions that generate several stable states. Those enable to go from one stable state to another by bringing energy. This is achieved, as will be explained further below in detail, by the change of a resonance frequency which maximizes the available/conducted energy.

### Embodiments

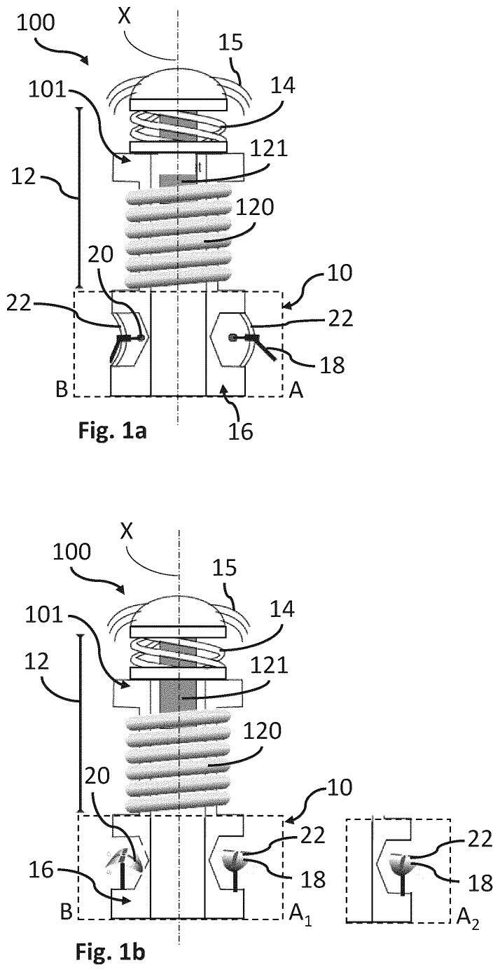

As can be seen on FIGS. 1a and 1b, the steering system 10 according to the present invention, is aimed at being part of a micro-robot 100 configured to move inside a fluidic environment, more precisely along a propulsion direction following a propulsion axis X. This movement along the propulsion direction happens by vibration inside the fluidic environment.

The micro-robot 100 thus comprises a body 101 comprising an actuator 12 configured to generate vibrations. More precisely, in the represented embodiments, the actuator 12 is a vibrating motor 12 which comprises a coil 120 and a magnet 121. The coil 120 extends along the propulsion direction X and surrounds the magnet 121. The magnet 121 is activable by the coil 120 and is configured to move back and forth along the propulsion direction X. The movement of the magnet 121 induces a compression/decompression movement of a propulsion spring 14 also part of the vibrating motor 12 and also extending along the propulsion direction X. Said propulsion spring 14 allows the movement of a series of external pilis or cilia 15 which enable the micro-robot 100 to be put in motion inside the fluidic environment.

Many fluidic environments can be targeted, but specifically all bodily fluids are concerned. Those bodily fluid are, for example, blood, cerebra spinal fluids, urine, bile liquid, lymph fluid, aqueous humor. Those fluids all present a viscosity close to that of water.

As can be further seen on FIGS. 1a and 1b, the steering system 10 according to the present invention comprises an elongated resonating structure 16 aimed at being secured to the micro-robot 100.

This elongated resonating structure 16 comprises:
a steering structure 18 aimed at controlling the propulsion direction,
a distribution of weight-resonators 20 configured to be activated over a proper (or given) activation resonance frequency f_{A}, f_{A1}, f_{A2} and deactivated under a proper (or given) deactivation frequency f_{B}.

The respective proper activation resonance frequencies f_{A}, f_{A1}, f_{A2} of each weight-resonator 20 is different from the proper activation resonance frequencies f_{A}, f_{A1}, f_{A2} of the other weight-resonators 20. The actuator 12 is configured to generate vibrations in a range of frequencies including the proper activation resonance frequency f_{A}, f_{A1}, f_{A2} of each weight-resonator 20. The elongated resonating structure 16 displays at least two states:
at least one activated steering state A, A₁, A₂, in which the configuration and movement of the elongated resonating structure 16 are configured to change the propulsion direction of the micro-robot 100,
a non-activated steering state B, in which the configuration and movement of the elongated resonating structure 16 are configured to maintain the propulsion direction of the micro-robot 100.

Regardless of the embodiment, the state B, A, A₁, A₂ of the elongated resonating structure 16, is determined by the activation or deactivation of the weight-resonators 20 as will be explained further below.

As will be explained further below, the activation and deactivation frequencies f_{B}, f_{A}, f_{A1}, f_{A2} of the weight-resonator 20 are induced by the vibrations of the micro-robot body 101 (mor particularly the vibrations induced by the micro-motor comprised within the body 101 of the micro-robot) when the micro-robot 100 is moving along its propulsion direction.

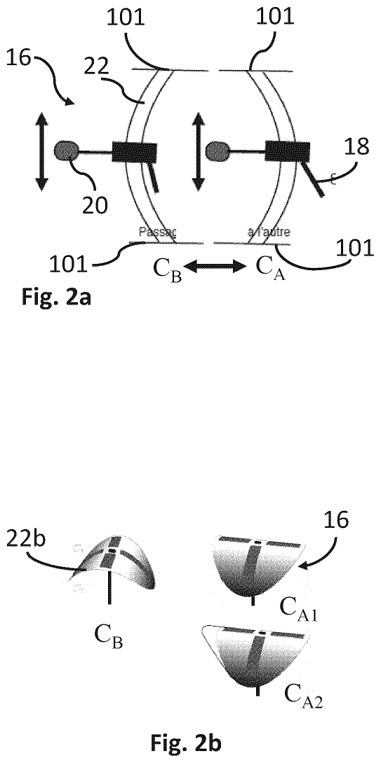

More particularly regarding FIGS. 1a and 1b, the elongated resonating structure 16 further comprises a distribution of multi-stable element 22 secured to the micro-robot 100. Each multi-stable element 22 displays at least two stable configurations and is deformable between those at least two stable configurations:
at least a first stable configuration C_{A}, C_{A1}, C_{A2} when the resonating structure 16 is in its activated steering state A, A₁, A₂ (see FIGS. 2a, 2b),
at least a second stable configuration C_{B} when the resonating structure 16 is in its non-activated steering state B (see FIGS. 2a, 2b).

Depending on the embodiments, the multi-stable element 22, the weight-resonator 20 and the steering structure 18 can be the same technical element or distinct elements. For example, in the embodiment depicted on FIG. 1a, the multi-stable element 22, the weight resonator 20 and the steering structure 18 are all distinct technical elements. However, regarding the embodiment depicted on FIG. 1b, the multi-stable element 22, the weight-resonator 20 and the steering structure 18 are the same technical element. In this case, the different proper activation frequencies f_{A}, f_{A1}, f_{A2} could be dedicated to, for example, the control the shape of the weight-resonator 20 (or multi-stable element 22). One could have a frequency for semi-closure, another for full closure, for example.

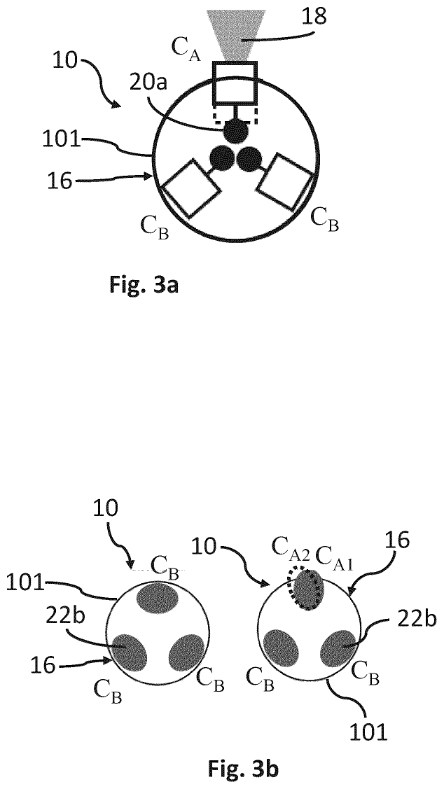

More precisely regarding the embodiment depicted on FIGS. 1a, 2a and 3a, the elongated resonating structure 16 comprises three multi-stable elements 22, equidistantly distributed around the body 101 of the micro-robot 100 (see FIG. 3a). Each multi-stable elements 22 is associated to a weight-resonator 20 having a specific proper activation frequency f_{A}. This specific proper activation frequency f_{A} being different from the proper activation frequencies f_{A} of the other weight-resonator 20 associated to the other multi-stable elements 22. In this example, each multi-stable element 22 is a bi-stable pre-compressed beam 22 between 100 μm and a few mm in length, made of polymers, glass or metal such as stainless steel or alliage. In this particular embodiment, each beam 22 connects the steering structure 18 and the weight-resonator 20. Each beam 22 has two extremities, and each extremity is secured to the micro-robot 100. More precisely, each beam 22 is secured in a small open cavity of the body 101 of the micro-robot 100. As already mentioned, each beam 22 presents a first stable configuration C_{A} in which it bends inwards the cavity of the micro-robot 100 and a second stable configuration C_{B} in which it bends outwards the cavity of the micro-robot 100. The first stable configuration C_{A} corresponds to the activated steering state A, the second stable configuration C_{B} corresponds to the non-activated steering state B. In its non-activated steering state B, the pre-compressed beam 22 is bent in a first direction, away from the body 101 of the micro-robot 100. In its activated steering state A, the pre-compressed beam 22 is bent in a second direction, towards the body 101 of the micro-robot 100 and thus different from the first direction.

Still considering the embodiment on FIGS. 1a and 2a, the steering structure 18 comprises a retractable steering foil 18 displaying an open and a retracted configuration with regards to the body 101 of the micro-robot 100 (see FIG. 2a). As will be explained later, the configuration of the steering foil 18 is determined by the state of the elongated resonating structure 16 of the steering system 10. The foils could be made of many different flexible materials such as metals such as copper or alliage metals, glass or polymers among others. Size can vary from about 100 μm to a few mm. More precisely, the non-activated steering state B of the elongated resonating structure 16 induces a retracted configuration of the retractable steering foil 18. On the other hand, the activated steering state A of the elongated resonating structure 16 induces an open configuration of the retractable steering foil 18.

Still considering FIG. 1a, each weight-resonator 20 comprises a resonating mass connected to the beam 22. This mass can be of any suitable shape, for example a bead or a cube. Depending on the embodiment the resonating mass could also be embedded in the beam 22 by extra thickness or an extension of the shape of the beam 22. The activation of the resonating mass of the weight-resonator 20 thus induces a configuration change in the beam 22. The configuration change of the beam 22a further induces a configuration change of the corresponding steering foil 18.

In the embodiment presented in FIGS. 1b, 2b and 3b, the multi-stable element 22 is a multi-stable shell 22 comprising a stack of several sheets. The multi-stable elements could be made of many different flexible materials who have this multi-stable state capacity when shaped appropriately such as copper. Size can vary from about 100 μm to a few mm.

In this embodiment, the multi-stable shell 22 displays a non-activated steering state B, in this state none of the shells oppose a resistance to the movement of the system by blocking the flow of the liquid, and several activated steering states A₁, A₂. Each activated steering state A₁, A₂ is associated to a different proper activation resonance frequency f₁, f₂. In those activated states, a shell is positioned in a way that oppose the flow, this creates an asymmetry and induces a rotation in the direction to which the shell is exposed.

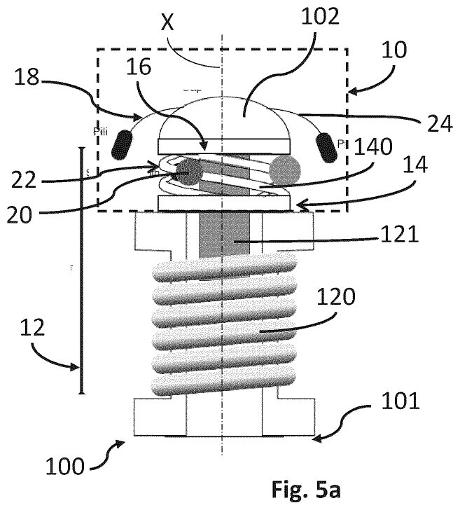

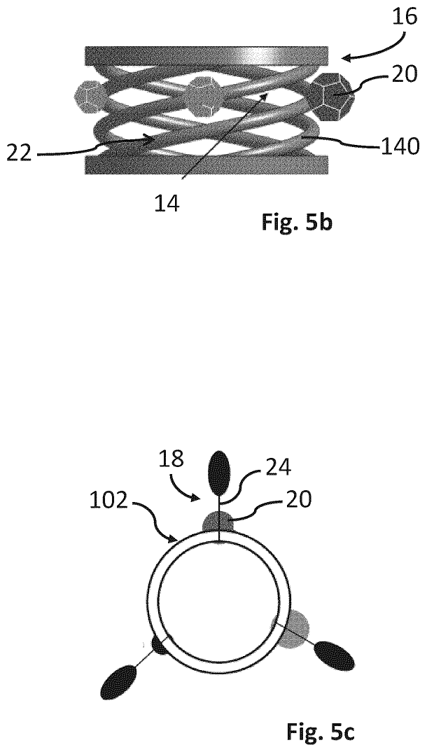

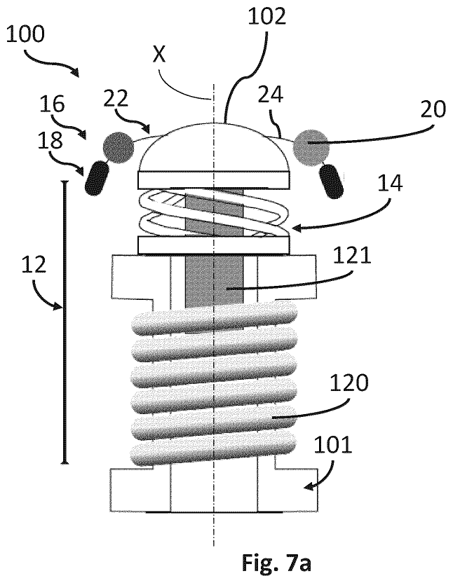

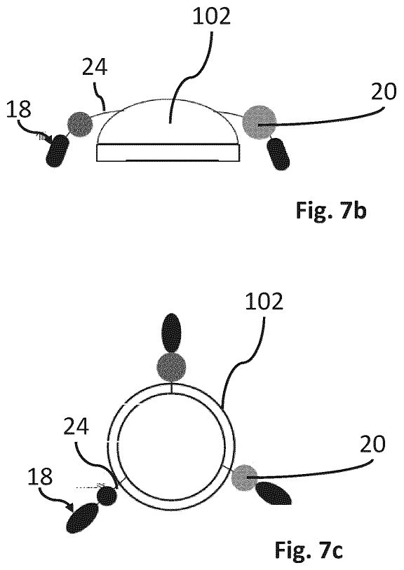

According to further embodiments depicted, in particular, on FIGS. 5a and 7a, the steering system 10, and more particularly the elongated resonating structure 16, comprises at least one mobile cilium 24. In some embodiment, the elongated resonating structure 16 comprises at least one, preferably a group of mobile cilia 24. This at least one mobile cilium 24 can be part of the series of external pilis or cilia 15 which enable the micro-robot 100 to be put in motion inside the fluidic environment (see paragraph [009]). The at least one mobile cilium 24 may also be an independent technical element from said series of external pilis or cilia 15.

In the embodiments of FIGS. 5a and 7a, the at least one cilium 24 is part of the steering structure 18. In this embodiment, the steering structure further is configured to enable a preferential activation of one single cilium 24 over the other cilia 24. In order to achieve this, the steering structure may comprise a foil carried by said at least one cilium 24. The foil can present different orientations depending on the configuration and/or position of the at least one cilium 24. Those different configurations/positions enable the steering structure 18 to control the propulsion direction of the micro-robot 100.

Each cilium 24 is secured to the body 101 of the micro-robot 100, preferably on the head portion of the micro-robot 100. More precisely, each 24 is secured to a mobile part 102 of the body 101 of the micro-robot 100. This mobile part 102 is connected to the propulsion spring 14 of the vibrating motor 12 and moves in accordance with said propulsion spring 14. Depending on the movements of the propulsion spring 14, said mobile part 102 can be centred or off-centred around the propulsion direction X. Each cilium 24 is thus configured to be put in motion by the vibration of the micro-robot 100, more particularly by the movement of the propulsion spring 14 of the motor (actuator) 12.

In this embodiment, the propulsion string 14 comprises several strands 140 from which some are equipped weight-resonators 20 (see FIG. 5b). Thus, in this embodiment, the elongated resonating structure 16 comprises at least one strand 140 of the propulsion spring 14 of the vibrating motor/actuator 12.

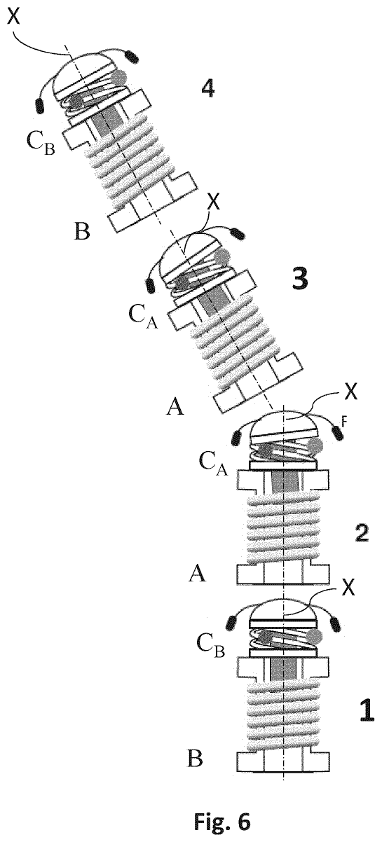

More particularly, in the embodiment depicted on FIGS. 5a, 5b, 5c and 6, each strand 140 carrying a weight-resonator 20 is part of the multi-stable element 22 of the steering system 10. Each weight-resonator 20 is, similarly to the precedingly detailed embodiments, activable over a proper activation resonance frequency f_{A}, f_{A1}, f_{A2} and deactivated under a given deactivation frequency f_{B}. As can be seen on FIG. 5b, the propulsion spring 14 comprises three independent strands 140 and each strand 140 carries a weight-resonator 20 which is activable over a different proper activation frequency f_{A}, f_{A1}, f_{A2}. As hinted at in FIG. 5b, the different weight-resonator 20 present different sizes and shapes, leading to different proper activation frequencies f_{A}, f_{A1}, f_{A2}. In an alternative embodiment, the different proper activation frequencies f_{A}, f_{A1}, f_{A2} could be dedicated to, for example, the control the shape of the weight-resonator 20. One could have a frequency for semi-closure, another for full closure.

In this embodiment (FIGS. 5a, 5b, 5c and 6), the activation of the elongated resonating structure 16 in its at least one activated steering state A, A₁, A₂ (see FIG. 6), and more particularly the activation of each weight-resonator 20 induces a retractation of the corresponding strand 140 of the propulsion spring 14.

More particularly, in this embodiment (FIGS. 5a, 5b, 5c and 6), each strand 140 presents a first stable configuration C_{A} in which it presents a first length L_{A} corresponding to an activated steering state A₁, A₂ and a second stable configuration C_{B} in which it presents a second length L_{B}, corresponding to the non-activated steering state B. When the strand 140 is in its second stable configuration C_{B}, its length L_{B} is the same than the length of the other strands 140. In this second stable configuration, the mobile part 102 of the body 101 of the micro-robot 100, is centred with regards to an elongation axis X of the micro-robot 100. Said elongation axis X is the same as the propulsion axis X already mentioned previously. When the strand 140 is in its activated configuration C_{A}, its length changes and the mobile part 102 of the body 101 of the micro-robot 100 is off-centred. The activation of the elongated resonating structure 16 in one of its activated steering states A₁, A₂ thus induces a break in the global symmetry of the body 101 of the micro-robot 100.

Regarding the embodiments depicted respectively on FIGS. 1a, 2a, 3a and 1b, 2b and 3b, it can also be said that the activation of the elongated resonating structure 16 in one of its activated steering states A₁, A₂ induces a break in the global symmetry of the body 101 of the micro-robot 100: the spreading of the foils 18a, 18b break the global rotational symmetry of the micro-robot 100.

The length of each strand 140 depends on the activation/inactivation of each associated weight-resonator 20. The length of each strand 140 thus depends on the vibration amplitude of the vibrating motor 12 and of the propulsion spring 14, going from about 0 (it does not move) to an amplitude in hundreds of um.

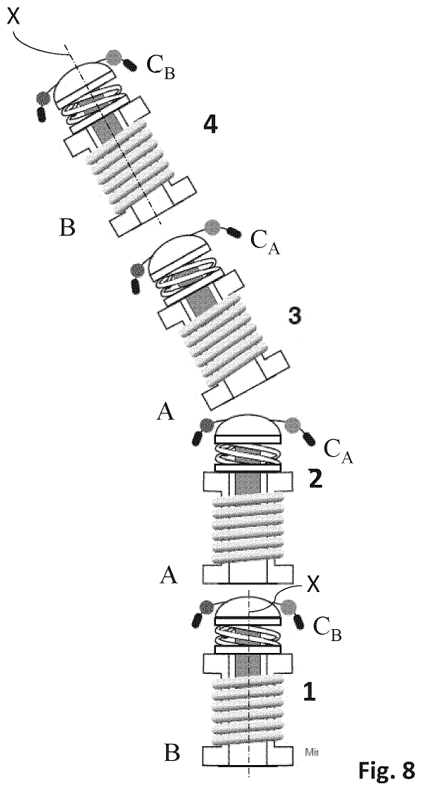

In the last depicted embodiment of the present invention (FIGS. 7a, 7b, 7c and 8), the at least one cilium 24 is also part of the elongated resonating structure 16. However, it is also part of the steering structure 18 and of the multi-stable element 22. As in the previous embodiment, each cilium 24 carries a foil in order to enable some steering of the micro-robot 100.

In this embodiment, the weight-resonator 20 activable by the frequencies of the propulsion spring 14, are carried by the cilia 24. More particularly, each cilium 24 carries a weight-resonator 20 activable at a propre activation frequency f_{A}, f_{A1}, f_{A2}.

In this embodiment, the at least one cilium 24 presents at least two stable states C_{A}, C_{B}, each stable state C_{A}, C_{B} being associated to a movement intensity. This way, each cilium 24 presents a first stable state C_{B} correspondent to a first movement intensity I_{B}. This first stable state C_{B} corresponds to the non-activated state of the weight-resonator 20 carried by the cilium 24 and thus corresponds to the non-activated steering state B. The second stable state C_{A} corresponds to a second movement intensity I_{A} and thus to the activated steering state A. The second movement intensity I_{A} being higher than the first movement intensity I_{B}. All cilium 24 in the first stable state C_{B} are moving (back and forth movements) with the same first movement intensity I_{B}. Once one (or several) cilium (cilia) 24 is (are) activated, it (they) start(s) moving with a different intensity, the second movement intensity I_{A}, thus inducing a break in the general symmetry of the micro-robot 100 which leads to a disequilibrium and eventually to a direction change.

### Functioning

As mentioned above, for each embodiment, the activation of the weight-resonator 20 induces the elongated resonating structure 16 to enter its at least one activated steering state A, A₁, A₂. On the other hand, the deactivation of the weight-resonator 20 induces the elongated resonating structure 16 to enter its non-activated steering state B.

When the elongated resonating structure 16 is in its non-activated steering state B, the propulsion direction of the micro-robot 100 is maintained the same and the micro-robot 100 moves straight forward along the propulsion axis X. However, when the elongated resonating structure 16 enters its at least one activated steering state A, A₁, A₂, the micro-robot 100 rotates and changes its propulsion direction.

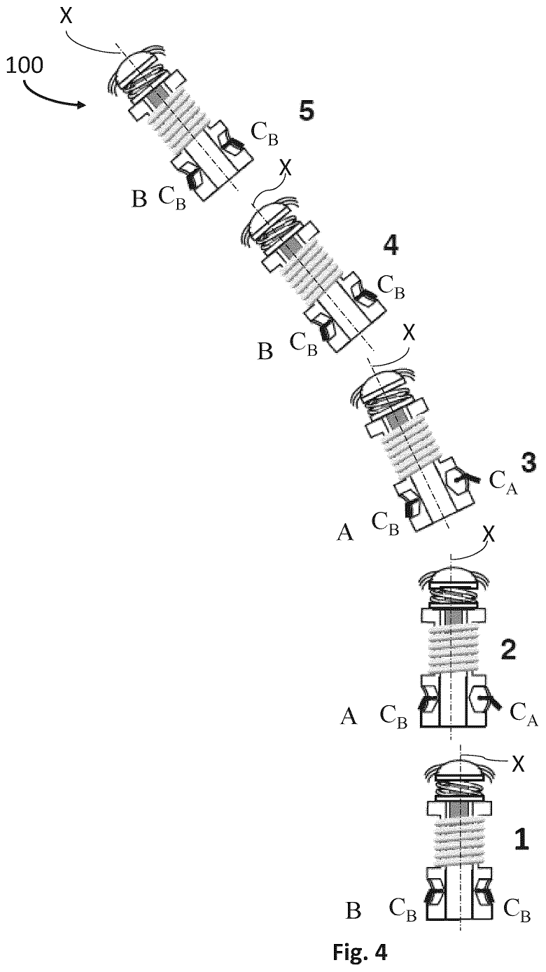

Considering the first embodiment (FIGS. 1a, 2a, 3a, 4), the vibration of the propulsion spring 14 induces the micro-robot 100 to vibrate at given frequencies. When the micro-robot 100 vibrates at the proper activation resonance frequency f_{A} of one of the weight-resonators 20 of the elongated resonating structure 16, the considered weight-resonator 20 starts vibrating. This vibration induces a configuration change in its associated pre-compressed beam 22a from its inactivated configuration C_{B} to its activated configuration C_{A}. This configuration change leads the associated steering foil 18 to be expanded. The elongated resonating structure 16 finds itself in its steering activated state A. The expansion of the steering foil 18 induces a rotation of the micro-robot 100 and a redefinition of the propulsion direction X (see FIG. 6). When the vibration of the micro-robot 100 falls below the given deactivation frequency f_{B} of the weight-resonator 20, the weight-resonator 20 is deactivated and the pre-compressed beam 22 falls back into its inactivated configuration C_{B}. The steering foil 18 is thus retracted and the elongated resonating structure 16 falls back in its inactivated steering state B. The frequency and/or amplitude of the motor 12 is changed to go from one resonant frequency related to a specific state to another related to another state.

Regarding the second embodiment (FIGS. 1b, 2b, 3b), the functioning is similar to the precedent embodiment, with exception that the vibrating of the propulsion spring 14 activates directly the multi-stable shell 22b, which changes configuration and induces the micro-robot 100 to rotate.

Considering the fourth embodiment (FIGS. 5a, 5b, 5c, 6), it has to be specified that each cilium 24 vibrates at a given intensity, said vibration being induced by the vibrations of the vibrating motor (actuator) 12 of the micro-robot 100. When the micro-robot 100 vibrates at the proper activation resonance frequency f_{A} of one of the weight-resonators 20 of the elongated resonating structure 16, the considered weight-resonator 20 starts vibrating. This vibration induces a configuration change in its associated strand 140, more precisely a modification of its length from its second length L_{B} defining its inactivated state C_{B}, to its first length L_{A} defining its activated state C_{A}. This change in length induces a break in the global symmetry of the body 101 of the micro-robot 100 and the at least one cilium 24 undergoes a shift in its vibration axis, thus inducing a rotation of the micro-robot 100 (see FIG. 6).

Regarding the last embodiment (FIGS. 7a, 7b, 7c and 8), when the micro-robot 100 vibrates at the proper activation resonance frequency f_{A} of one of the weight-resonators 20 of the elongated resonating structure 16, the considered weight-resonator 20 starts vibrating. This vibration induces a configuration change in the vibration intensity of the associated cilium 24. The cilium 24 then changes from its first movement intensity I_{B} (corresponding to the steering non-activated state B of the elongated resonating structure 16) to its movement intensity I_{A} (corresponding to the steering activated state A of the elongated resonating structure 16). The activated cilium 24 thus vibrates at a different speed and a different amplitude from the other cilia 24 (or the series of external pilis or cilia 15) and this induces a rotation of the micro-robot 100 (see FIG. 7).

This rotation of the micro-robot 100 most likely happen by a succession of jolts, creating a discrete cumulation of several small rotational movements leading to the desired final rotation. This enables an additional layer of precision and security.

The fact of being able to manage the 3D orientation of a device with only one linear actuator on which a user can change the activation frequency from a distance, gives a 2D plane of solutions (one axis=frequency, one axis=intensity) and one need solely to navigate in this plane to have the desired configuration, which is both more easy and more reliable.

1-13. (canceled)

14. A Micro-robot configured to move along a propulsion direction by vibrations,
said micro-robot comprising a body configured to vibrate, and an actuator configured to generate vibrations causing the micro-robot to move,
said micro-robot further comprising a steering system which comprises a resonating structure configured to be secured to the micro-robot, the resonating structure comprising:
a steering structure aimed at controlling the propulsion direction,
a distribution of weight-resonators, each weight-resonator being configured to be activated by a proper activation resonance frequency, the respective proper activation resonance frequencies of the weight-resonators being different from each other,
wherein the actuator is configured to generate vibrations in a range of frequencies including the proper activation resonance frequency of each weight-resonator,
wherein the resonating structure displays at least two states:
at least one activated steering state, in which at least one of the weight-resonators is activated at the proper activation resonance frequency to change the propulsion direction of the micro-robot,
a non-activated steering state, in which none of the weight-resonators is activated at its proper activation resonance frequency so as to maintain the propulsion direction of the micro-robot.

15. The micro-robot according to claim 14, wherein the resonating structure displays several activated steering states, each activated steering state being associated to a different proper activation resonance frequency.

16. The micro-robot according to claim 14, wherein:
the at least one activated steering state, is a state in which the configuration and movement of the resonating structure aim at changing the propulsion direction of the micro-robot,
the non-activated steering state, is a state in which the configuration and movement of the resonating structure aim at maintaining the propulsion direction of the micro-robot.

17. The micro-robot according to claim 14, wherein the resonating structure further comprises a distribution of multi-stable elements, each multi-stable element being deformable between at least two stable configurations:
at least a first stable configuration when the resonating structure is in its activated steering state,
at least a second stable configuration when the resonating structure is in its non-activated steering state.

18. The micro-robot according to claim 17, wherein each multi-stable element is a bi-stable pre-compressed beam displaying the first and the second stable configurations, the pre-compressed beam being bent in a first direction towards the body of the micro-robot in the first stable configuration and being bent in a second direction away from the body of the micro-robot in the second stable configuration.

19. The micro-robot according to claim 14, wherein the steering structure is a retractable steering foil displaying an open and a retracted configuration with regards to the body of the micro-robot, the steering foil configuration being determined by the steering state of the resonating structure:
the non-activated steering state of the resonating structure induces the retracted configuration of the retractable steering foil, and
the activated steering state of the resonating structure induces the open configuration of the retractable steering foil.

20. The micro-robot according to claim 17, wherein each multi-stable element is a multi-stable shell comprising a stack of several sheets.

21. The micro-robot according to claim 14, wherein the steering system further comprises at least one mobile cilium, the at least one cilium being configured to be put in motion by the vibration of the body of the micro-robot.

22. The micro-robot according to claim 21, wherein the resonating structure comprises at least one strand of a propulsion spring comprised within the body.

23. The micro-robot according to the precedent claim 22, wherein the activation of the resonating structure in its at least one activated steering state induces the retractation of the at least one strand of the propulsion spring.

24. The micro-robot according to claim 22, wherein the body of the micro-robot presents a global circular symmetry along a propulsion axis parallel to the propulsion direction, wherein the resonating structure is further part of said body, and wherein the activation of the resonating structure into one of its activated steering states induces a break in the global circular symmetry of the body.

25. The micro-robot according to claim 21, wherein the at least one cilium is part of the resonating structure, the at least one cilium presenting a first movement intensity in the non-activated steering state and a second movement intensity in the activated steering state, the second movement intensity being different than the first movement intensity.

26. The micro-robot according to claim 25, wherein each cilium comprises a weight-resonator.
